(19) United States
(12) Patent Application Publication
(10) Pub. No.: US 2013/0245526 A1
(43) Pub. Date: Sep. 19, 2013

(54) BANDAGE AND METHOD OF USING SAME

(71) Applicant: Ronald F. Marascalco, Oxford, MS (US)

(72) Inventor: Ronald F. Marascalco, Oxford, MS (US)

(21) Appl. No.: 13/423,330

(22) Filed: Mar. 19, 2012

(51) Int. Cl. A61F 13/02; A61L 15/26; A61L 15/22; A61F 13/56; A61L 15/20

(52) U.S. Cl. 602/43; 604/385.03; 604/375; 604/369; 604/372

(57) ABSTRACT

A bandage for covering a wound treated with an allograft can include a liquid permeable skin adherent layer having a wound facing surface with an adhesive thereon for adhering to skin proximate the wound. A compressible support section can be supported by the skin adherent layer for maintaining the allograft in place on the wound, and an absorbent section can be positioned over the compressible support section. The absorbent section can be comprised of a liquid absorbent material and can aid in keeping the allograft hydrated. The compressible support section can be positioned intermediate the skin adherent layer and the absorbent section. A liquid permeable covering layer can be positioned over the absorbent section, and attached to the skin adherent layer.

### TECHNICAL FIELD AND BACKGROUND OF THE INVENTION

The present invention relates to a bandage that can be used to cover a wound. One embodiment of the invention comprises a bandage that can cover an ulceration on the human epidermis being treated with an allograft, and maintain the allograft in place without the use of sutures.

The transplantation of tissue, known as allografts, can be used to treat and heal ulcerated tissue on the human epidermis. For example, one allograft, sold under the trademark EPIFIX, comprises an amniotic membrane that facilitates tissue regeneration of ulcerated tissue on the epidermal surface. Typically, the allograft is positioned over the ulcerated tissue and held in place by sutures. A conventional moisture retentive dressing is then positioned over the allograft sutured to the wound to keep out external contaminants, and keep the allograft hydrated. The optimal environment for a healing wound is a moist environment having a temperature close to normal body temperature. The temperature of an open wound is typically lower than the body's normal temperature as body heat escapes through the wound opening. Reduced wound temperature can result in physiologic effects that impair wound healing.

The process of suturing the allograft and applying a moisture retentive dressing typically takes thirty minutes to an hour. With ever increasing demands on the time of health care practitioners and to reduce the costs of health care services, there is a need for an apparatus and method that would reduce the time and cost for treating a wound on the epidermis with an allograft.

### SUMMARY OF THE INVENTION

Therefore, one object of the present invention is to provide a bandage that can be used to cover an allograft positioned on a wound on the epidermis. Another object of the present invention is to provide a bandage that can maintain the allograft in place on the wound without the need for sutures. Yet another object of the invention is to provide a bandage that helps retain heat in the wound.

These and other objects of the present invention can be achieved in preferred embodiments of the invention described below. One embodiment of the invention comprises a bandage comprising a skin adherent layer having a wound facing surface having an adherent thereon for attaching to epidermal tissue. A compressible support section is supported by the skin adherent layer, and an absorbent section comprised of liquid absorbent material is supported by the compressible support section. The compressible support section is positioned intermediate the skin adherent layer and the absorbent section, and a liquid permeable covering layer is positioned over the absorbent section and functionally connected to the skin adherent layer.

According to another embodiment of the invention, a fluid impermeable layer is positioned intermediate the compressible support section and the absorbent section.

According to another embodiment of the invention, the fluid impermeable layer comprises cellophane.

According to another embodiment of the invention, a second fluid impermeable layer comprised of cellophane is positioned intermediate the skin adherent layer and the compressible support section.

According to another embodiment of the invention, a fluid impermeable layer comprising cellophane encloses the compressible support section, and prevents external fluid from penetrating the support section. The fluid impermeable layer also prevents fluid that is hydrating the absorbent section from escaping from the bandage and leaking onto surrounding areas, such as clothes, bedding and/or furniture.

According to another embodiment of the invention, the compressible support section is comprised of foam, a sponge, and/or a gauze pad.

According to another embodiment of the invention, the absorbent section is comprised of cotton and/or a super absorbent polymer, such as polyacrylic acid.

According to another embodiment of the invention, the absorbent section includes bacteriostatic material.

According to another embodiment of the invention, the adherent comprises an adhesive adapted for adhering to epidermal tissue.

According to another embodiment of the invention, the skin adherent layer comprises a stretchable and liquid permeable material.

According to another embodiment of the invention, the skin adherent layer has a surface opposite the wound facing surface with no adherent thereon.

According to another embodiment of the invention, a bandage for covering a wound treated with an allograft comprises a liquid permeable skin adherent layer having a wound facing surface having an adhesive thereon for adhering to skin proximate the wound. A support section is supported on the skin adherent layer. The support section is comprised of a material that maintains the allograft in place on the wound and prevents the escape of body heat through the wound. An absorbent section is positioned over the support section. The absorbent section is comprised of a liquid absorbent material, and the support section is positioned intermediate the skin adherent layer and the absorbent section. A liquid permeable covering layer is positioned over the absorbent section, and attached to the skin adherent layer. The absorbent section aids in keeping the allograft hydrated.

According to another embodiment of the invention, a fluid impermeable layer encloses the compressible support section. As such, the fluid impermeable layer protects the support section against penetration of external fluid.

According to another embodiment of the invention, the fluid impermeable layer comprises cellophane.

According to another embodiment of the invention, the absorbent section includes bacteriostatic water.

According to another embodiment of the invention, a chamber is positioned intermediate the support section and the absorbent section. The chamber can contain fluid for hydrating the absorbent section, and has at least one opening for allowing the fluid contained in the chamber to flow into the absorbent section.

According to another embodiment of the invention, the fluid contained within the chamber can be bacteriostatic water and/or sterile saline. A removable cover can cover the chamber opening(s) so that removing the cover exposes chamber opening(s) to allow the bacteriostatic water and/or sterile saline in the chamber to flow into the absorbent section.

According to another embodiment of the invention, a method of covering a wound treated with an allograft includes providing a bandage comprising a liquid permeable skin adherent layer having a wound facing surface having an adhesive thereon adapted for adhering to epidermal tissue, and a compressible support section supported by the skin adherent layer for supporting and maintaining the allograft in place on the wound. A fluid impermeable layer encloses the compressible support section, and an absorbent section is supported by the compressible support section, and is comprised of a liquid absorbent material. The compressible support section is positioned intermediate the skin adherent layer and the absorbent section, and a liquid permeable outer covering layer extends over the absorbent section and the compressible support section and is functionally connected to the skin adherent layer. The outer covering layer is positioned against the allograft, whereby the support section maintains the allograft in place on the wound and gives structural support enabling the absorbent section to reach the full depth of the wound.

According to another embodiment of the invention, the wound facing surface of the skin adherent layer is positioned on skin proximate the wound, so that the bandage is adhered to the skin.

### BRIEF DESCRIPTION OF THE DRAWINGS

FIG. 1 is a top plan view of a bandage according to a preferred embodiment of the invention;

FIG. 2 is a cross sectional view of the bandage taken along lines 2-2 of FIG. 1;

FIG. 3 is an environmental perspective view illustrating a method of using the bandage of FIG. 1;

FIG. 4 is cross sectional environmental view illustrating a method of using the bandage of FIG. 1; and

FIG. 5 is a cross sectional view of a bandage according to another preferred embodiment of the invention.

### DESCRIPTION OF THE PREFERRED EMBODIMENTS OF THE INVENTION AND BEST MODE

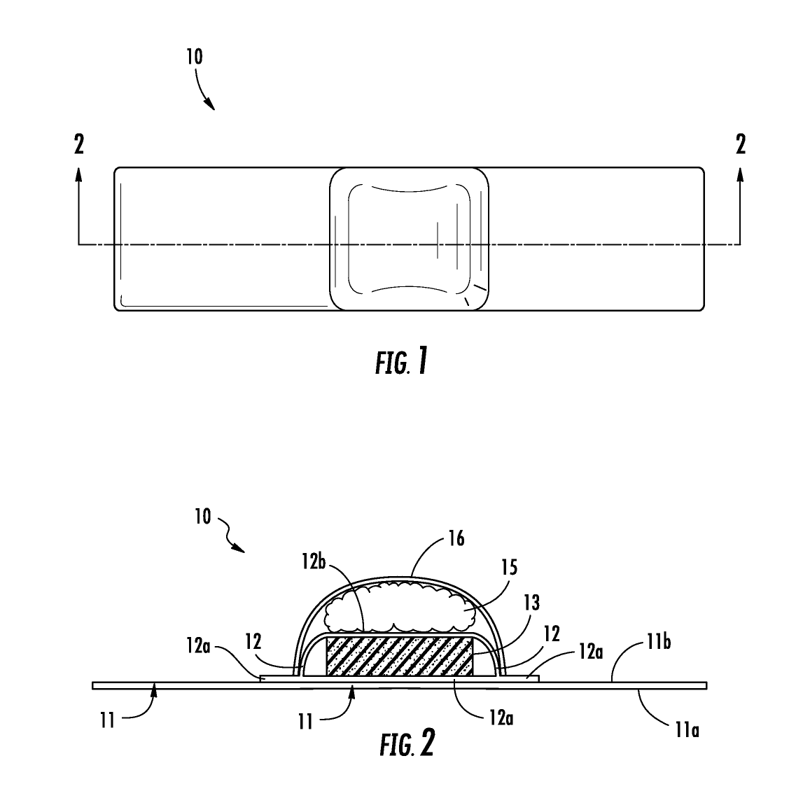

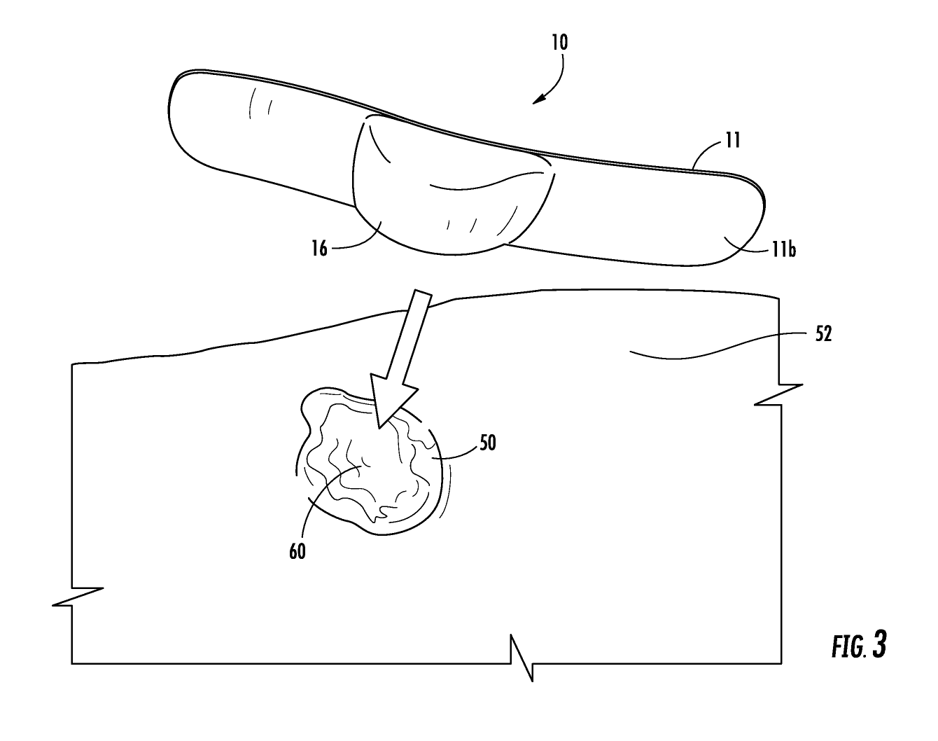

A bandage according to a preferred embodiment of the invention is illustrated in FIGS. 1 and 2, and shown generally at reference numeral 10. As shown in FIG. 1, the bandage 10 comprises a skin adherent layer 11 having an outer surface 11a, and an inner wound facing surface 11b opposite the outer surface 11a. The skin adherent layer 11 is comprised of a strip of stretchable, liquid permeable material. The wound facing surface 11b has an adhesive adapted for adhering to epidermal tissue. The outer surface 11a has no adhesive. Alternatively, a peelable cover can be positioned on the wound facing surface 11b to cover and keep the adhesive clean until use when the cover can be peeled off to expose the adhesive.

As shown in FIG. 2, a support section 13 is positioned on a center area of the wound facing surface 11b of the skin adherent strip 11. The support section 13 can be comprised of a material that is compressible yet firm, and acts as a heat shield that can retain heat, such as a memory foam pad. Alternatively, the support section 13 can be comprised of a sponge, gauze pad, or any other suitable material that can provide the appropriate structural support.

The support section 13 can be enclosed by a fluid impermeable layer 12 that includes a first section 12a positioned between the support section 13 and the skin adherent layer 11, and a second section 12b covering the opposite side of the support section 13. The fluid impermeable layer 12 can be comprised of cellophane. Alternatively, instead of fully enveloping the support section 13 with a fluid impermeable layer 12, only a first layer of cellophane 12a can be positioned between the support section 13 and the skin adherent strip 11, and a second layer 12b of cellophane can be positioned between the support section 13 and an absorbent section 15.

The absorbent section 15 is positioned on the compressible support section 13, and comprised of a liquid absorbent material. The absorbent section 15 can be comprised of cotton, a super absorbent polymer, such as polyacrylic acid, and/or any other suitable material capable of retaining sterile liquid. The absorbent section 15 can also include bacteriostatic water contained within the liquid absorbent material.

A covering layer 16 has first and second ends attached to the portion of the fluid impermeable layer 12a that is attached to the wound facing surface 11b of the skin adherent layer 11, as shown in FIG. 2. The covering layer 16 extends over the absorbent section 15 and the support section 13, and keeps the absorbent section 15 and support section 13 in place on the skin adherent strip 11. The covering layer 16 can be comprised of a liquid permeable material.

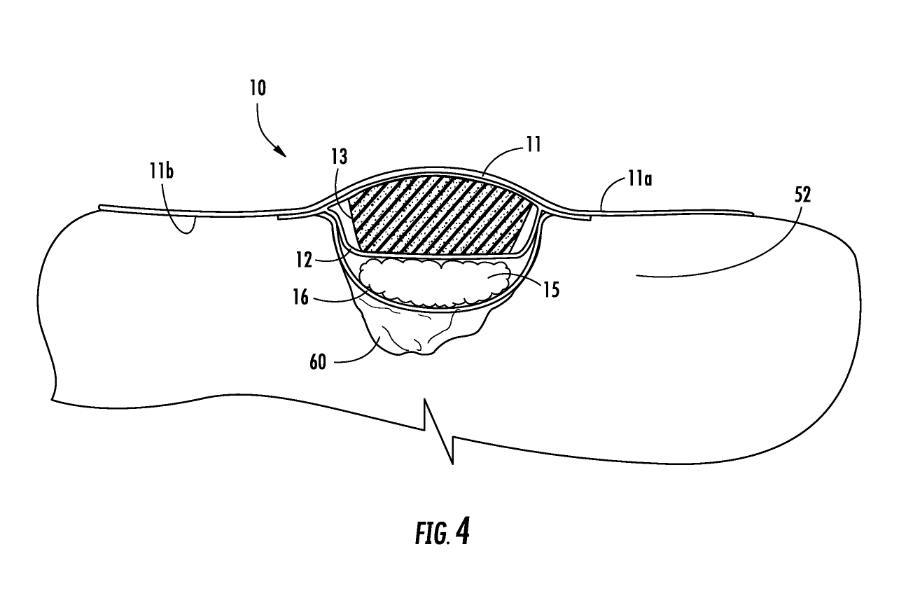

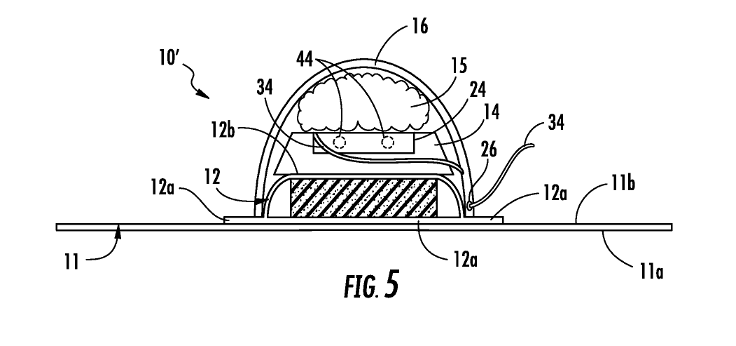

FIGS. 3 and 4 illustrate a method of using the bandage 10 according to a preferred embodiment of the invention, in which the bandage 10 can be used to cover a wound such as a skin ulceration 50 being treated with an allograft 60. As shown in FIG. 4, the outer covering layer 16 is positioned against the allograft 60, and the wound facing surface 11b of the skin adherent strip 11 is adhered to skin 52 proximate the wound 50 and allograft 60. The liquid permeable covering layer 16 allows for bacteriostatic water or other desired liquid contained in the absorbent section 15 to penetrate and keep the allograft hydrated. The pressure exerted by the compressible support section 13 on the allograft 60 keeps the allograft in place on the wound, eliminating the need for sutures to keep the allograft in place on the wound. In addition, the support section 13 provides structural support that enables the absorbent section 15 to reach the full depth of the wound 50. Furthermore, the support structure 13 helps prevent the escape of body heat through the wound 50. As such, the bandage 10 aids in providing a moist environment and a wound temperature close to normal body temperature for optimal wound healing conditions. The bandage 10 can save significant time for the health care practitioner by eliminating the steps of suturing the allograft to the wound, and applying a moisture retentive dressing.

A bandage according to an alternative embodiment of the invention is illustrated in FIG. 5, and shown generally at reference numeral 10′. The bandage 10′ is similar in structure to the previously described embodiment, bandage 10, except for a fluid filled chamber 14 positioned between the support section 13 and the absorbent section 15. The chamber 14 can hold sterile saline, bacteriostatic water or other suitable fluid for hydrating the absorbent section 15. The chamber 14 can include a plurality of openings 44 that are covered by a removable cover strip 24. A string 34 can be attached to the cover 24, and exits through an opening 26 in the covering layer 16. A user can pull the string 34 to remove the cover 24, thereby exposing the chamber openings 44 to allow the fluid within the chamber 24 to flow into the absorbent section 15 keeping the absorbent section 15 hydrated. The chamber 14 can be a plastic bladder that has relatively little volume when emptied of fluid. The removable cover 24 can be comprised of plastic or other suitable material. The bandage 10′ can be otherwise identical in structure to the previously described embodiment, bandage 10.

A bandage and a method of using same are described above. Various changes can be made to the invention without departing from its scope. The above description of the preferred embodiments and best mode of the invention are provided for the purpose of illustration only and not limitation—the invention being defined by the following claims and equivalents thereof.

What is claimed is:

1. A bandage comprising:
(a) a skin adherent layer having a wound facing surface having an adherent thereon for attaching to epidermal tissue;
(b) a compressible support section supported by the skin adherent layer;
(c) an absorbent section supported by the compressible support section, and comprised of a liquid absorbent material, wherein the compressible support section is positioned intermediate the skin adherent layer and the absorbent section; and
(d) a liquid permeable covering layer positioned over the absorbent section and functionally connected to the skin adherent layer.

2. A bandage according to claim 1, further comprising a fluid impermeable layer positioned intermediate the compressible support section and the absorbent section.

3. A bandage according to claim 2, wherein the fluid impermeable layer comprises cellophane.

4. A bandage according to claim 2, further comprising a second fluid impermeable layer positioned intermediate the skin adherent layer and the compressible support section.

5. A bandage according to claim 4, wherein the first and second fluid impermeable layers comprise cellophane.

6. A bandage according to claim 1, further comprising a fluid impermeable layer enclosing the compressible support section, whereby the fluid impermeable layer prevents external fluid from penetrating the support section.

7. A bandage according to claim 6, wherein the fluid impermeable layer comprises cellophane.

8. A bandage according to claim 1, wherein the compressible support section comprises one or more selected from the group consisting of foam, a sponge, and a gauze pad.

9. A bandage according to claim 1, wherein the absorbent section comprises one or more selected from the group consisting of cotton and polyacrylic acid.

10. A bandage according to claim 1, wherein the absorbent section comprises one or more selected from the group consisting of bacteriostatic material and sterile material.

11. A bandage according to claim 1, wherein the adherent comprises an adhesive adapted for adhering to epidermal tissue.

12. A bandage according to claim 1, wherein the skin adherent layer comprises a liquid permeable material.

13. A bandage according to claim 1, wherein the skin adherent layer includes a surface opposite the wound facing surface having no adherent thereon.

14. A bandage for covering a wound treated with an allograft comprising:
(a) a liquid permeable skin adherent layer having a wound facing surface having an adhesive thereon for adhering to skin proximate the wound;
(b) a support section supported by the skin adherent layer adapted for maintaining the allograft in place on the wound and preventing the escape of body heat through the wound;
(c) an absorbent section supported by the support section, and comprised of a liquid absorbent material, wherein the support section is positioned intermediate the skin adherent layer and the absorbent section and provides structural support to the absorbent section urging the absorbent section; and
(d) a liquid permeable covering layer positioned over the absorbent section, and attached to the skin adherent layer, wherein the absorbent section aids in keeping the allograft hydrated.

15. A bandage according to claim 14, further comprising a fluid impermeable layer enclosing the support section, whereby the fluid impermeable layer protects the support section against penetration of external fluid.

16. A bandage according to claim 14, further comprising a chamber positioned intermediate the support section and the absorbent section containing fluid for hydrating the absorbent section, the chamber having at least one opening for allowing fluid contained in the chamber to flow into the absorbent section.

17. A bandage according to claim 16, wherein the chamber contains one or more fluids selected from the group consisting of bacteriostatic water and sterile saline, and includes a removable cover for covering the at least one opening whereby removing the cover exposes the at least one opening to allow the fluid contained in the chamber to flow into the absorbent section.

18. A bandage according to claim 14, wherein the support section comprises a compressible material, and the absorbent section comprises one or more selected from the group consisting of cotton and polyacrylic acid.

19. A method of covering a wound treated with an allograft comprising the steps of:
(a) providing a bandage comprising:
(i) a liquid permeable skin adherent layer having a wound facing surface having an adhesive thereon adapted for adhering to epidermal tissue,
(ii) a compressible support section supported by the skin adherent layer for supporting and maintaining the allograft in place on the wound,
(iii) a fluid impermeable layer enclosing the compressible support section,
(iv) an absorbent section supported by the compressible support section, and comprised of a liquid absorbent material, wherein the compressible support section is positioned intermediate the skin adherent layer and the absorbent section, and
(v) a liquid permeable outer covering layer positioned over the absorbent section and functionally connected to the skin adherent layer; and
(b) positioning the outer covering layer against the allograft, whereby the support section maintains the allograft in place on the wound and the absorbent section keeps the allograft hydrated.

20. A method according to claim 19, further comprising the step of positioning the wound facing surface of the skin adherent layer on skin proximate the wound, whereby the bandage is adhered to the skin.
